(19) United States
(12) Patent Application Publication
(10) Pub. No.: US 2025/0302323 A1
(43) Pub. Date: Oct. 2, 2025

(54) ELECTRONIC DEVICE FOR MEASURING BIOMETRIC INFORMATION AND OPERATING METHOD OF SAME ELECTRONIC DEVICE

(71) Applicant: SAMSUNG ELECTRONICS CO., LTD., Suwon-si (KR)

(72) Inventors: Jeehoon LEE, Suwon-si (KR); Eungi MIN, Suwon-si (KR); Inho YUN, Suwon-si (KR)

(21) Appl. No.: 19/238,326

(22) Filed: Jun. 13, 2025

Related U.S. Application Data
(63) Continuation of application No. PCT/KR2023/020619, filed on Dec. 14, 2023.

(30) Foreign Application Priority Data
Dec. 14, 2022 (KR) 10-2022-0174454
Jan. 19, 2023 (KR) 10-2023-0008228

(51) Int. Cl. A61B 5/024; A61B 5/11

(52) U.S. Cl. CPC A61B 5/02427; A61B 5/02438; A61B 5/1123

(57) ABSTRACT

An electronic device for measuring a biometric signal and an operating method of the electronic device is provided. The electronic device may set a plurality of operating modes for a sensor module and may set operating information for each of the plurality of operating modes, identify an operating mode to be used on the basis of a designated event occurrence, identify a voltage of the sensor module to be applied to a plurality of light-emitting devices on the basis of the operating information set in the identified operating mode. The electronic device may further output the identified voltage, where the identified voltage is less than reference voltage, and obtain biometric information on the basis of light irradiated from each of the plurality of light-emitting devices according to the identified voltage.

### CROSS-REFERENCE TO RELATED APPLICATIONS

This application is a continuation of International Application No. PCT/KR2023/020619 designating the United States, filed on Dec. 14, 2023, in the Korean Intellectual Property Receiving Office and claiming priority to Korean Patent Application Nos. 10-2022-0174454, filed on Dec. 14, 2022, and 10-2023-0008228, filed on Jan. 19, 2023, in the Korean Intellectual Property Office, the disclosures of each of which are incorporated by reference herein in their entireties.

### TECHNICAL FIELD

Embodiments of the disclosure relate to an electronic device for measuring biometric information and a method of operating the electronic device.

### BACKGROUND ART

Recently, electronic devices have been developed in various forms for the convenience of users, and have been miniaturized so that users may conveniently carry them.

Interest in health and exercise to maintain health has increased in the recent years. Accordingly, electronic devices have been developed in various forms to measure and use various biosignals of the human body, and provide various services to manage the health of users or check their health condition through the measurement of various biosignals.

The above information is presented as related art only to assist with an understanding of the disclosure. No determination has been made, and no assertion is made, as to whether any of the above might be applicable as prior art with regard to the disclosure.

### DETAILED DESCRIPTION OF THE INVENTION

A conventional electronic device uses the same application voltage for an optical sensor in each operation mode, and unnecessarily consumes power in an operation mode that may be performed with less power, since the same application voltage is also used in other operation modes according to an operation mode in which the most current (LED current) is used in light emitting elements of the optical sensor.

The disclosure provides an electronic device and method for measuring biometric information by changing an application voltage for at least one optical sensor according to a set operation mode, thereby reducing the power consumption of measuring the biometric information.

According to an embodiment of the disclosure, an electronic device may include a sensor module including a plurality of light emitting elements and at least one optical sensor, wherein the plurality of light emitting elements include a first light emitting element and at least one of a second light emitting element or a third light emitting element, memory storing instructions, and at least one processor.

According to an embodiment, the instructions, when executed by the at least one processor, cause the electronic device to set a plurality of operation modes for the sensor module and to set operation information for each of the plurality of operation modes.

According to an embodiment, the instructions, when executed by the at least one processor, cause the electronic device to, based on occurrence of a specified event, identify an operation mode to be used,

According to an embodiment, the instructions, when executed by the at least one processor, cause the electronic device to, based on the operation information set for the identified operation mode, identify a voltage to be applied to the plurality of light emitting elements and output the identified voltage. The identified voltage may be less than a reference voltage.

According to an embodiment, the instructions, when executed by the at least one processor, may cause the electronic device to obtain biometric information based on light irradiated from each of the plurality of light emitting elements to which the identified voltage is applied.

According to an embodiment of the disclosure, a method of operating an electronic device may include setting a plurality of operation modes for a sensor module of the electronic device and operation information for each of the plurality of operation modes.

According to an embodiment, the method may include, based on occurrence of a specified event, identifying an operation mode to be used.

According to an embodiment, the method may include, based on the operation information set for the identified operation mode, identifying a voltage to be applied to the plurality of light emitting elements and outputting the identified voltage. The identified voltage may be less than a reference voltage.

According to an embodiment, the method may include obtaining biometric information based on light irradiated from each of the plurality of light emitting elements to which the identified voltage is applied. According to an embodiment, the plurality of light emitting elements may include a first light emitting element and at least one of a second light emitting element or a third light emitting element.

According to an embodiment of the disclosure, in a non-transitory storage medium storing a program, the program may include executable instructions that, when executed by at least one processor of an electronic device, cause the electronic device to set a plurality of operation modes for a sensor module of the electronic device and operation information for each of the plurality of operation modes, based on occurrence of a specified event, identify an operation mode to be used, based on the operation information set for the identified operation mode, identify a voltage to be applied to the plurality of light emitting elements and output the identified voltage, wherein the identified voltage is less than a reference voltage, and obtain biometric information based on light irradiated from each of the plurality of light emitting elements to which the identified voltage is applied. According to an embodiment, the plurality of light emitting elements may include a first light emitting element and at least one of a second light emitting element or a third light emitting element.

### BRIEF DESCRIPTION OF DRAWINGS

FIG. 1 is a block diagram illustrating an electronic device in a network environment according to various embodiments.

FIG. 2 is a block diagram illustrating the configuration of an electronic device according to an embodiment.

FIGS. 3A and 3B are diagrams illustrating an example of the configuration of an electronic device according to an embodiment.

FIG. 4 is a block diagram illustrating a sensor module of an electronic device according to an embodiment.

FIGS. 5A and 5B are diagrams illustrating examples of voltages applied to a plurality of light emitting elements included in a sensor module of an electronic device according to an embodiment.

FIGS. 6A, 6B and 6C are diagrams illustrating exemplary operations of a sensor module in an electronic device according to an embodiment.

FIG. 7 is a diagram illustrating an example of setting voltages to be applied to a plurality of light emitting elements in an electronic device according to an embodiment.

FIG. 8 is a diagram illustrating an exemplary method of operating an electronic device according to an embodiment.

FIG. 9 is a diagram illustrating an example of improved power consumption of an electronic device according to an embodiment.

FIG. 10 is a diagram illustrating an example of improved power consumption of an electronic device according to an embodiment.

In connection with the description of the drawings, the same or similar reference numerals may be used for the same or similar components.

### MODE FOR CARRYING OUT THE INVENTION

An electronic device according to various embodiments will be described below with reference to the attached drawings. The term user used in various embodiments may refer to a person using an electronic device or a device (e.g., an artificial intelligence electronic device) using an electronic device.

An embodiment of the disclosure will be described below in detail with reference to the drawings so that those skilled in the art may easily practice the disclosure. However, the disclosure may be implemented in various different forms and is not limited to the embodiment described herein. In relation to the description of the drawings, the same or similar reference numerals may be used for the same or similar components. In addition, in the drawings and related descriptions, a description of well-known functions and configurations will be avoided for clarity and conciseness.

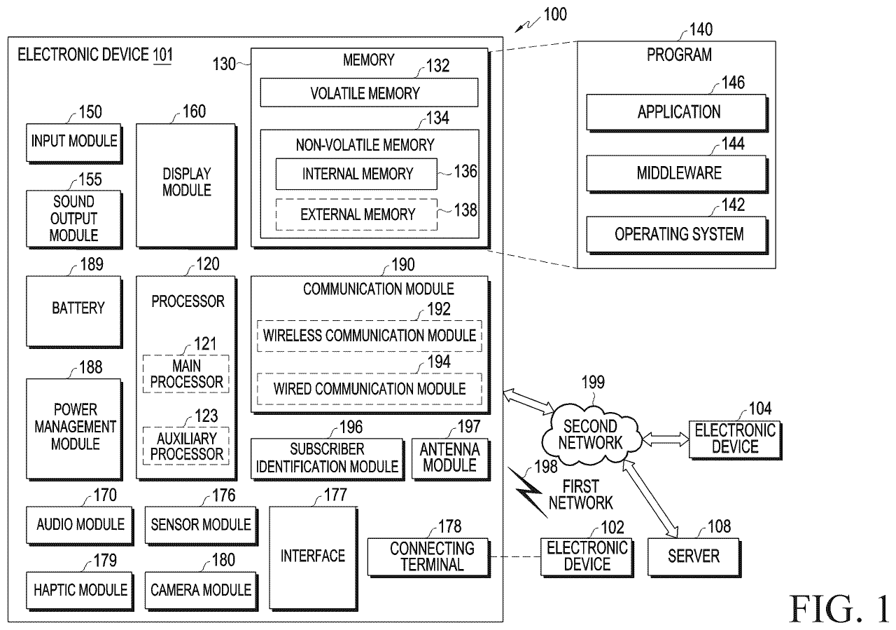

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the strength of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form an mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The above-described electronic device 101 of FIG. 1 may be an electronic device for measuring biometric information or an external electronic device connected to the electronic device for measuring biometric information. According to an embodiment of the disclosure, the electronic device 101 of FIG. 1 will be described as an example of the electronic device for measuring biometric information.

The electronic device (e.g., the electronic device 101 of FIG. 1) of the disclosure may be implemented in various forms for measuring biometric information. The electronic device may include various sensors capable of measuring various pieces of biometric information such as a user's heart rate (or pulse rate), blood oxygen saturation, stress, and blood pressure by using various sensors. The various sensors include, but are not limited to, an optical sensor such as, for example, a photoplethysmography (PPG) sensor, and a motion sensor. In one or more non-limiting embodiments, the electronic device may be implemented in the form of a wearable device that may be worn on the user's body to measure a biosignal of the user's body part. The electronic device may measure various pieces of biometric information of the user by using signals (e.g., biosignals) detected by sensors. The biometric information described in the disclosure may be described as health information or other terms.

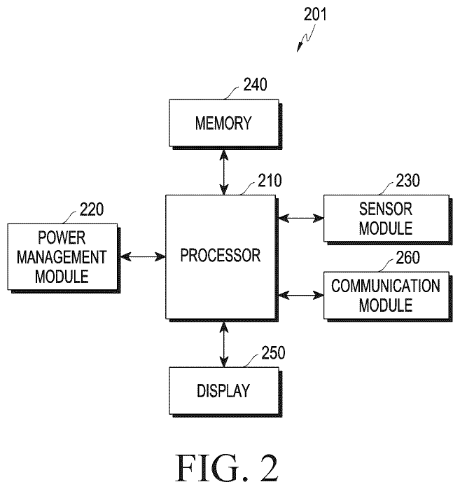

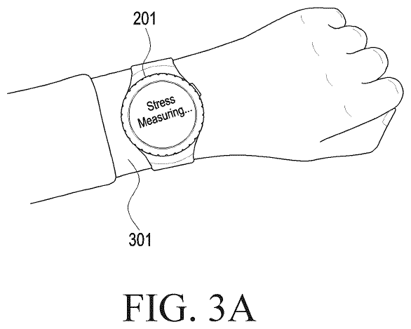

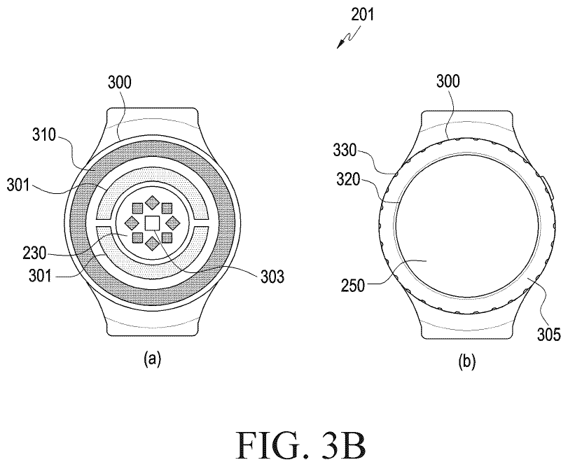

FIG. 2 is a block diagram illustrating the configuration of an electronic device according to an embodiment, and FIGS. 3A and 3B are diagrams illustrating an example of the configuration of an electronic device according to an embodiment.

Referring to FIG. 2, an electronic device 201 (e.g., the electronic device 101 of FIG. 1) according to an embodiment may be configured to include at least one processor 210 (e.g., the processor 120 of FIG. 1), a sensor module 230 (e.g., the sensor module 176 of FIG. 1), memory 240 (e.g., the memory 130 of FIG. 1), a display 250 (e.g., the display module 160 of FIG. 1), and/or a communication module 260 (e.g., the communication module 190 of FIG. 1). The electronic device 201 is not limited thereto and may be configured to further include an electrode module or various other components or to exclude some of the above components. According to an embodiment, the electronic device 201 may further include a power management module (or circuit) 220 (e.g., the power management module 188 of FIG. 1), and the power management module 220 may be configured separately from the sensor module 230 or included in the sensor module 230.

Referring to FIG. 3A, the electronic device 201 according to an embodiment may be a watch-type wearable device that may be worn on the user's wrist. For example, the electronic device 201 may be implemented in the form of a glasses type, a patch type, a ring type, or other various types of wearable devices that may be worn on other parts of the user's body (e.g., head, forearm, thigh, or other parts of the human body on which electrocardiograms are measurable).

Referring to FIG. 3B, the electronic device 201 according to an embodiment may include a housing 300, which includes a first surface 310 (e.g., rear surface), a second surface 320 (e.g., front surface), and a third surface 330 (e.g., side surface) that surrounds a space between the first surface 310 (e.g., rear surface) and the second surface 320 (e.g., front surface). According to an embodiment, in the electronic device 201, the sensor module 230 capable of measuring at least one biosignal may be disposed to come into contact with or be close to the skin of the human body, on a third member 303 formed in a form surrounded by a first member 301 disposed on the first surface 310 that is one surface of the housing 300.

Referring to FIG. 2 again, the processor 210 of the electronic device 201 according to an embodiment may set a plurality of operation modes for operations of the sensor module 230 and may also set operation information required for the operation of the sensor module 230 in each operation mode. The operation information may include information about a voltage to be applied to a plurality of light emitting elements included in the sensor module 230, and light information (e.g., the types of light emitting elements and/or an operating current (LED current), and operating voltage (e.g., current driver forward voltage (Vf), current driver headroom voltage (Vh), and margin voltage associated with each of the light emitting elements to be used among light emitting elements of different wavelengths. According to an embodiment, the processor 210 may set a plurality of operation modes that operate with different intensities based on a sensor measurement purpose, the type of biometric information, and/or the user's situation (e.g., movement situation or current movements), characteristic information about the user (e.g., the user's skin color), or a surrounding environment (e.g., on-demand measurement or automatic measurement, an ambient illuminance, or time). The processor 210 may set a first operation mode for measuring first biometric information (e.g., a heart rate) in a situation where the user's movement is low (e.g., normal times such as when the user is sitting or lying down, or standing with limited movement), a second operation mode for measuring second biometric information (e.g., a heart rate with a high current intensity or high intensity movements) in a situation (e.g., exercise) where the user's movement is so high as to be equal to or greater than a first threshold, and a third operation mode for measuring third biometric information (e.g., blood oxygen saturation) in a situation (e.g., sleep) where the user's movement is so low as to be equal to or less than a second threshold. In addition, the processor 210 may further set an operation mode for measuring biometric information such as stress and blood pressure.

According to an embodiment, the processor 210 may identify an operation mode to be used based on occurrence of a specified event, and identify an application voltage applied to a plurality of light emitting elements based on configured operation information in the identified operation mode. According to an embodiment, the processor 210 may irradiate light to the user's body part during a specified period by the plurality of light emitting elements to which the identified voltage is applied, and receive at least a portion of light reflected from the body part. According to an embodiment, the processor 210 may obtain a plurality of optical signals of different wavelengths from the light received during the specified period, and obtain biometric information based on the plurality of optical signals.

According to an embodiment, based on the biometric information, the processor 210 may obtain at least one of information related to the user's physical condition, information (e.g., warning information and/or guidance information) related to an abnormal sign of the user's health condition, or additional information (e.g., at least one of health training information, hospital information, emergency treatment information, or information for relieving stress) according to the user's physical condition or health condition.

According to an embodiment, the processor 210, which is a hardware module or a software module (e.g., an application program), may be a hardware component (function) or a software component (program), including at least one of various sensors provided in the electronic device 201, a data measurement module, an input/output interface, a module for managing the status or environment of the electronic device 201, or a communication module.

According to an embodiment, the processor 210 may include, for example, a combination of one or more of hardware, software, or firmware. The processor 210 may be configured to omit at least some of the above components, and/or to further include other components for performing an image processing operation in addition to the above components.

According to an embodiment, the power management module (or circuit) 220 may be electrically connected to the at least one processor 210 and the sensor module 230 or may be included in the sensor module 230. The power management module 220 may output an application voltage set for each operation mode to a plurality of light emitting elements of the sensor module 230 under the control of the at least one processor 210.

According to an embodiment, the sensor module (e.g., sensor circuit) 230 may include a plurality of light emitting elements and various sensors (e.g., an optical sensor (PPG sensor), a motion sensor, and/or an electrocardiogram (e.g., ECG) sensor) for detecting biometric information. The sensor module 230 may be configured to be electrically connected to the processor 210. According to an embodiment, the optical sensor (e.g., PPG sensor) included in the sensor module 230 detects a PPG signal to non-invasively measure oxygen saturation in the user's body part by using pulse oximetry, and the PPG signal (e.g., optical signal) is a signal measured from light reflected or transmitted after irradiating light to tissues and blood vessels, and may be used to measure a change in a blood flow caused by pulse waves.

The electronic device 201 according to an embodiment may further include a motion sensor (e.g., an accelerometer sensor, a gyroscope, a barometer, and/or a geomagnetic sensor) for detecting the movement of the user. The acceleration sensor may detect acceleration or impact caused by the movement of the electronic device 201 or the user carrying the electronic device 201. The gyroscope may detect the rotation direction or rotation angle of the electronic device 201 due to the movement of the electronic device 201 or the user carrying the electronic device 201. The barometric sensor may detect barometric pressure, and the geomagnetic sensor may detect the direction of a geomagnetic field. The user's motion (or movement) state may be identified using acceleration sensing information, gyro sensing information, barometric pressure sensing information, and/or geomagnetic sensing information detected from the motion sensor according to an embodiment. For example, the user's motion state may be identified as a state of no movement (e.g., stationary), a state of no movement or if any, weak movement (e.g., sedentary), or a state of movement (or a specified user activity state (e.g., walking or running)).

According to an embodiment, the sensor module 230 of the electronic device 201 may further include at least one other sensor for detecting a biosignal in addition to at least one optical sensor and at least one motion sensor. For example, the at least one other sensor (not shown) may include, but is not limited to, a body temperature sensor, an ECG sensor, an electrodermal activity (EDA) sensor, or/and a sweat sensor. The body temperature sensor according to an embodiment may measure the temperature of the body. The ECG sensor according to an embodiment may measure an electrocardiogram by detecting an electrical signal from the heart through electrodes attached to the body. The EDA sensor according to an embodiment may include, for example, a galvanic skin response (GSR) sensor, and measure the user's excitement state by detecting a skin electrical activity. The sweat sensor according to an embodiment may detect sweat of the user's body and measure hydration and/or dehydration. At least one biometric sensor according to an embodiment may detect a biosignal of the user under the control of the processor 210 and provide information (a value or a numerical value) (e.g., skin temperature, electrocardiogram, stress, skin conductivity, hydration, and/or dehydration) based on the measured biosignal to the processor 210.

According to an embodiment, the memory 240 (e.g., the memory 130 of FIG. 1) according to an embodiment may store information and/or data related to the operation of the electronic device 201. The memory 240 according to an embodiment may store instructions that, when executed by the electronic device 101, cause the processor 210 to perform the operation described above. The memory 240 (e.g., the memory 130 of FIG. 1) may store an application (software code, algorithm, function or program) related to biometric information measurement, information related to an operation mode set for biometric information measurement, and biometric information measured in each mode. According to an embodiment, the memory 240 may store various applications (e.g., an exercise application, a health management application, or a medical-related application) in addition to the application related to biometric information measurement. According to an embodiment, the memory 240 may store various data generated during program execution, including a program (e.g., the program 140 of FIG. 1) used for a functional operation. The memory 240 may largely include a program area (e.g., the program 140 of FIG. 1) and a data area (not shown). The program area 140 may store related program information for driving the electronic device 201, such as an operating system (OS) (e.g., the operating system 142 of FIG. 1) that boots the electronic device 201. The data area (not shown) may store transmitted and/or received data and generated data according to an embodiment. Further, the memory 240 may be configured to include, as a storage medium, at least one of flash memory, a hard disk, a multimedia card micro type memory (e.g., secure digital (SD) or extreme digital (XD) memory), RAM, or ROM. According to an embodiment, the memory 240 may store biometric information and operation information configured for each operation mode.

According to an embodiment, the display 250 may display biometric information measured in each operation mode and information related to health or exercise obtained based on the biometric information. According to an embodiment, the display 250 may display guidance information related to the execution of the operation for measuring biometric information in each operation mode. According to an embodiment, the display 250 may display information (e.g., warning information and/or guidance information) related to an abnormal sign of the user's health condition or additional information (e.g., at least one of health training information, hospital information, emergency treatment information, or information for relieving stress) according to the user's physical condition or health condition, based on the biometric information. According to an embodiment, the display 250 may be implemented in the form of a touch screen. When the display 250 is implemented with an input module in the form of a touch screen, it may display various pieces of information generated according to the user's touch action. According to an embodiment, the display 250 may be configured with at least one of a liquid crystal display (LCD), a thin film transistor LCD (TFT-LCD), an organic light emitting diode (OLED), a light emitting diode (LED), an active matrix organic LED (AMOLED), a micro LED, a mini LED, a flexible display, or a 3-dimensional display. In addition, some of these displays may be configured as a transparent type or a light-transmitting type so that the outside may be viewed through them. This may be configured in the form of a transparent display including a transparent OLED (TOLED). According to an embodiment, another display module (e.g., an extended display or a flexible display) may be further included in addition to the display 250.

According to an embodiment, the communication module (or circuit) 260 may communicate with an external electronic device (e.g., the electronic device 102 or 104 of FIG. 1, the server 108 of FIG. 1, or an electronic device of another user). For example, the communication module 260 may transmit measured biometric information to the external electronic device. According to an embodiment, the communication module 260 may perform at least one of cellular communication, ultra wide band (UWB) communication, Bluetooth communication, and/or wireless fidelity (WiFi) communication, and may further perform communication using other communication methods available for communication with external electronic devices. According to an embodiment, the communication module 260 may transmit at least one of information related to the user's physical condition obtained based on the biometric information by the at least one processor 210, information (e.g., warning information and/or guidance information) related to an abnormal sign of the user's health condition, or additional information (e.g., at least one of health training information, hospital information, emergency treatment information, or stress relief information) according to the user's physical condition or health condition to the external electronic device.

According to an embodiment, the electronic device 201 is not limited to the configuration illustrated in FIG. 2 and may be configured to further include various components.

According to an embodiment, the electronic device 201 may further include an audio module (not shown) (e.g., the audio module 170 of FIG. 1) or a vibration module (not shown) (e.g., the haptic module 179 of FIG. 1). The audio module may output sound and may be configured to include, for example, at least one of an audio codec, a microphone MIC, a receiver, an earphone output EAR_L, or a speaker. The audio module may output information related to the user's physical condition obtained based on biometric information, information related to an abnormal sign of the user's health condition, or additional information as an audio signal. For example, the vibration module may output information related to the user's physical condition obtained based on the biometric information, the information related to the abnormal sign of the user's health condition, or the additional information as vibration.

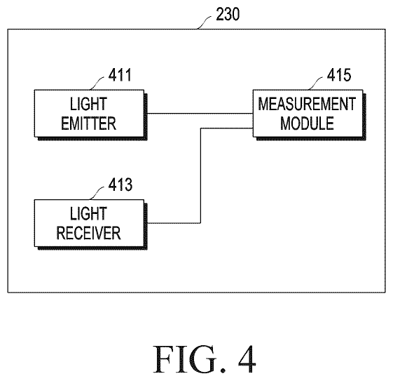

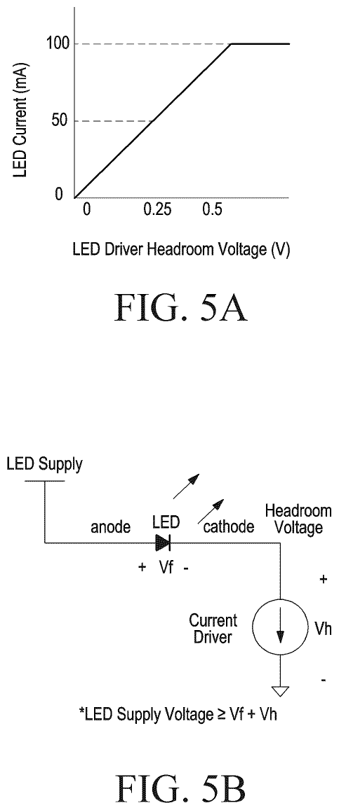

FIG. 4 is a block diagram illustrating a sensor module of an electronic device according to an embodiment, and FIGS. 5A and 5B are diagrams illustrating an example of voltages applied to a plurality of light emitting elements included in a sensor module of an electronic device according to an embodiment.

Referring to FIGS. 4, 5A and 5B, according to an embodiment, the sensor module (e.g., the sensor module 230 of FIG. 2) may include a light emitter 411 including a plurality of light emitting elements that output (e.g., emit) light of different wavelengths, a light receiver 413, and a measurement module 415. The measurement module 415 may include a photodiode (PD) that converts light energy into electrical energy. Without being limited thereto, the sensor module 230 may further include a signal processor (not shown) (e.g., an analog front end). The signal processor (not shown) may include an amplifier for amplifying a biosignal and an analog-to-digital converter (ADC) for converting an analog biosignal into a digital biosignal. However, the components included in the signal processor are not limited to the above-described amplifier and ADC.

According to an embodiment, the sensor module 230 may include a PPG sensor, and the PPG sensor may measure a change in blood volume in blood vessels by measuring the amount of reflected light using an optical sensor based on the characteristic that the volume of blood vessels changes due to a blood flow in peripheral blood vessels that changes as the heart repeatedly contracts and relaxes.

According to an embodiment, each of the plurality of light emitting elements may output light of a different wavelength externally therefrom. The output light is irradiated to the user's body, and at least a portion of the irradiated light may be reflected by a part 301 (e.g., skin, skin tissue, fat layer, vein, artery, or capillary) of the user's body. According to an embodiment, the sensor module 230 may receive at least a portion of the light reflected from the part 301 of the user's body through the light receiver 413, convert the light received by the light receiver 413 into an electrical signal through the measurement module 415, obtain a plurality of optical signals (e.g., biosignals) of different wavelengths, and output the obtained plurality of optical signals to at least one hardware component (e.g., the processor 120 of FIG. 1 or the processor 210 of FIG. 2) of an electronic device (e.g., the electronic device 201 of FIGS. 2 to 3B).

According to an embodiment, the sensor module 230 may be configured as at least one array. According to an embodiment, when there are a plurality of optical sensors, different weights may be applied to biosignals obtained from the plurality of optical sensors. According to an embodiment, the sensor module 230 may be disposed on the housing of the electronic device 201 or may be disposed to be exposed to the outside through the housing.

According to an embodiment, the light emitter 411 may convert electrical energy into light energy. The light output from the light emitter 411 may include infrared (IR) rays and visible light (e.g., red light, blue light, and/or green light). According to an embodiment, the light emitter 411 may include a light emitting element (e.g., LED) corresponding to each of IR light and visible light (e.g., red light, blue light, and/or green light).

According to an embodiment, when light output from the light emitter 411 is irradiated onto the skin, the sensor module 230 may detect at least a portion of reflected light remaining after a portion of the light is partially absorbed by the skin, through the light receiver 413. When the sensor module 230 is in contact with the body, the amount of blood in the blood vessels increases during the systole of the heart, and thus the amount of light detected through the light receiver 413 may decrease, whereas the amount of blood in the blood vessels decreases during the diastole of the heart, and thus the amount of light detected through the light receiver 413 may increase. The measurement module 415 according to an embodiment may measure various types of biometric information, such as oxygen saturation, blood pressure, blood sugar, heart rate, and/or blood volume by processing a signal based on the amount of the reflected light detected through the light receiver 413.

According to an embodiment, the light emitter 411 may include at least one light emitting element among a spectrometer, a vertical cavity surface emitting laser (VCSEL), an LED, a white LED, and a white laser. For example, the light emitter 411 may output IR light and/or visible light (e.g., red light, green light, or blue light) through the spectrometer, the VCSEL, the LED, the white LED, or the white laser.

Referring to FIGS. 5A and 5B, according to an embodiment, at least one light emitting element (LED) included in a light emitter (e.g., the light emitter 411 of FIG. 4) may have a current intensity that varies depending on the amount of current flowing to an anode and a cathode, and the amount of the current may be controlled by a current driver in an analog front end (AFE). As illustrated in FIG. 5A, as the current increases, the forward voltage of the light emitting element may increase, and the forward voltage may have different characteristics depending on the type of the light emitting element 411. For example, when the current of the light emitting element is about 50 mA, the headroom voltage of a light emitting element driver (e.g., current driver) may increase to about 0.25V, and when the current of the light emitting element is about 100 mA, the headroom voltage of the light emitting element driver may increase to about 0.5V. The plurality of light emitting elements included in the light emitter 411 may be set to different wavelengths and different forward voltages, respectively. For example, a first light emitting element that outputs green light among the plurality of light emitting elements may be set to a wavelength of about 525 nm and a forward voltage of about 2.5V. For example, among the plurality of light emitting elements, a second light emitting element that outputs red light may be set to a wavelength of about 660 nm and a forward voltage of about 2.1V. For example, among the plurality of light emitting elements, a third light emitting element that outputs a reference (IR) light may be set to a wavelength of about 950 nm and a forward voltage of about 1.3V.

According to an embodiment, as illustrated in FIG. 5B, as the current (LED current) of a light emitting element increases, a headroom voltage required for the current driver may also increase. As the current increases, the headroom voltage of the current driver may generally increase linearly. A voltage applied to an anode may be set to be greater than the sum of a forward voltage (Vf) and a headroom voltage (Vh) of the current driver. A voltage applied to the plurality of light emitting elements 411 may increase as the current increases and decrease as the current decreases.

According to an embodiment, a different current may be set for the plurality of light emitting elements 411 in each operation mode. According to an embodiment, an operation mode requiring high accuracy (e.g., a situation in which there is a lot of movement, such as an exercise state, equal to or greater than the first threshold) uses a lot of current of the light emitting elements, and an operation mode in which measurement is performed in a state in which high accuracy is not required or there is little movement (e.g., a situation in which there is little movement, such as a sleep state, equal to or less than the second threshold) may use a small current of the light emitting elements, so that power unnecessarily consumed by at least one sensor may be reduced by flexibly applying a voltage to the light emitting elements in each set operation mode. A battery voltage of the electronic device may be set in a range of about 3.4V to about 4.4V, and a reference application voltage of the light emitting elements may be set to about 5V. According to an embodiment, the light emitting elements 411 may operate as a buck converter, when an application voltage less than the battery voltage is required, and may operate as a boost converter when an application voltage greater than the battery voltage is required. The buck converter may have higher efficiency than the boost converter.

Referring again to FIG. 4, the light receiver 413 according to an embodiment may receive (detect or sense) at least a portion of light which has been irradiated by the light emitter 411 and reflected from a part of the wearer's body (e.g., the body part 301 of FIG. 3A). For example, the light receiver 413 may convert optical energy (light energy) sensed by at least one light receiving element into electrical energy. The light receiver 413 may detect a first optical signal (e.g., a red optical signal or red light) of a first wavelength band, a second optical signal (e.g., an IR optical signal or infrared light) of a second wavelength band, and a third optical signal (e.g., a green optical signal or green light) of a third wavelength band. For example, the first wavelength band and the second wavelength band may be longer than the third wavelength band, and the longer wavelength bands may be more sensitive to movement than the shorter wavelength band. For example, during a light sensing operation, a noise component caused by the movement may be included in an optical signal sensed according to the movement. When light sensing is performed on the same movement, the first optical signal (e.g., the red optical signal) may include more noise components than the green optical signal due to the movement. The light receiver 413 may include at least one light receiving element. For example, the light receiver 413 may include at least one of an avalanche PD, a single-photon avalanche diode (SPAD), a PD, a photomultiplier tube (PMT), a charge coupled device (CCD), a complementary metal-oxide-semiconductor (CMOS) array, or a spectrometer. For example, the structure of the light receiver 413 may be a reflective or a transmissive type. According to an embodiment, as illustrated in FIG. 4, when IR light and visible light (e.g., red light, blue light, and/or green light) are output from one light emitter 411, the light receiver 413 may include at least one light receiving filter (not shown) for selecting a desired wavelength band. According to an embodiment, the measurement module 415 or integrated circuit (IC) may be electrically connected to the light emitter 411, the light receiver 413, and the processor (e.g., the processor 210 of FIG. 2). The measurement module 415 may measure a biosignal (e.g., an optical signal based on photoplethysmography) based on an electrical signal corresponding to light (e.g., at least a portion of reflected light) received by the light receiver 413. The measurement module 415 according to an embodiment may obtain the first optical signal (e.g., IR optical signal) based on an electrical signal corresponding to first light (e.g., at least a portion of reflected IR light) detected by the light receiver 413 and/or the third optical signal (e.g., red optical signal) based on an electrical signal corresponding to third light (e.g., at least a portion of reflected red light), and may obtain the second optical signal (e.g., green or blue optical signal) based on an electrical signal corresponding to second light (e.g., at least a portion of reflected green or blue light) detected by the light receiver 413. The measurement module 415 according to an embodiment may transmit or process a plurality of received optical signals (IR optical signal, red optical signal, and/or green optical signal) of different wavelengths to the processor 210.

According to an embodiment, the processor 210 of the electronic device 201 may control the power management module 220 to output a voltage to be applied to a plurality of light emitting elements based on operation information configured for each operation mode. The processor 210 may activate (turn on) at least two light emitting elements configured for each operation mode, irradiate light output from each of the at least two activated light emitting elements to the user's body part 301, and control the light emitting elements, so that at least a portion of light reflected from the user's body part is received by the light receiver 413, and a plurality of optical signals of different wavelengths are detected by the measurement module 415. The obtained plurality of optical signals of different wavelengths may be reflection-type PPG signals from light reflected or scattered from the user's body part. According to an embodiment, the processor 210 may repeatedly obtain a plurality of optical signals of different wavelengths during a specified period.

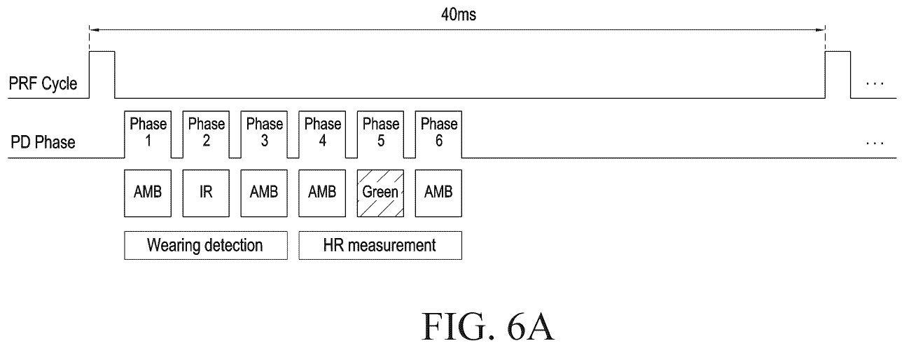

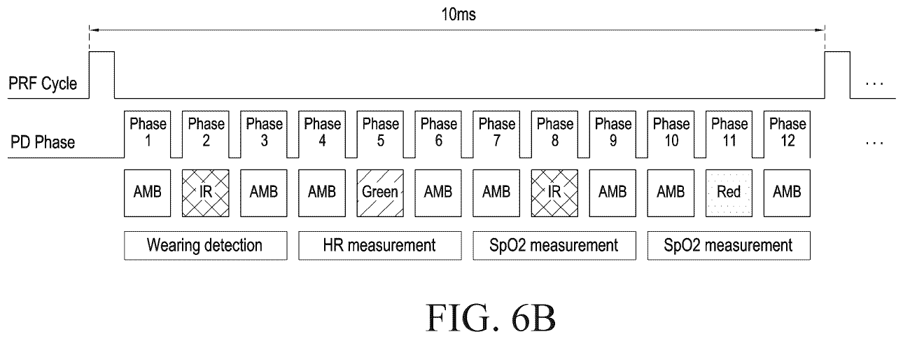

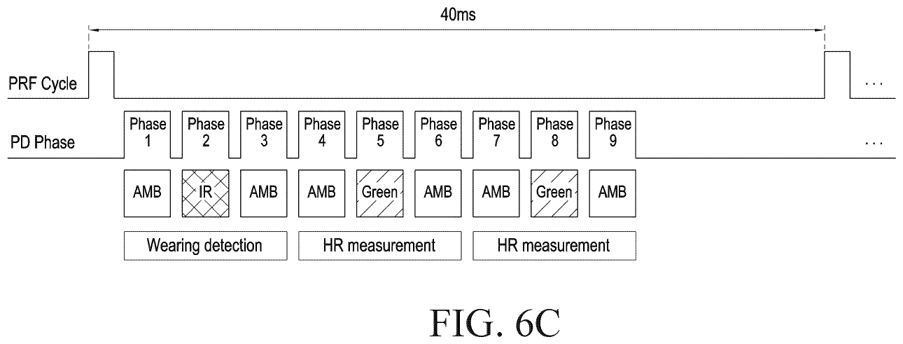

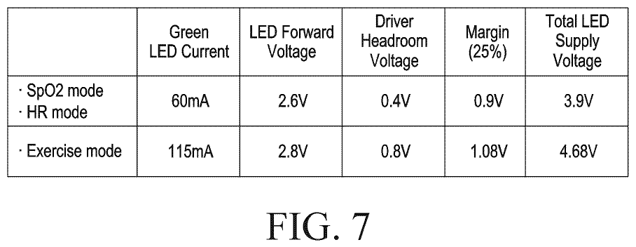

FIGS. 6A, 6B, and 6C are diagrams illustrating exemplary operations of a sensor module in an electronic device according to an embodiment. FIG. 7 is a diagram illustrating an example of setting a voltage to be applied to a plurality of light emitting elements in an electronic device according to an embodiment.

Upon occurrence of a specified event, a processor (e.g., the processor 210 of FIG. 2) of an electronic device (e.g., the electronic device 201 of FIGS. 2 to 3B) according to an embodiment may identify an operation mode for operating at least one optical sensor based on user movement information, environment information, and/or input information. The specified event may be an event based on detection of wearing of the electronic device, an input for requesting measurement of biometric information, a specified time, or execution of a specific application.

Referring to FIG. 6A, upon occurrence of a specified event, the processor 210 of the electronic device 201 according to an embodiment may obtain user movement information using the motion sensor, identify a situation (e.g., normal times) in which a user movement is less than the first threshold and equal to or greater than the second threshold based on the obtained movement information, and identify the operation mode of the sensor module 230 as the first operation mode, further based on environment information (e.g., time) of the user and/or input information in the situation (e.g., normal times) in which the movement is less than the first threshold and equal to or greater than the second threshold. The situation (e.g., normal time) in which the movement is less than the first threshold and equal to or greater than the second threshold may be a situation requiring low accuracy in measuring biometric information. For example, when a current time is daytime and the user movement is at the level of movement for daily life, the first operation mode (e.g., a mode for measuring a heart rate (HR) using a first current intensity (about 60 mA)) may be identified. According to an embodiment, the processor 210 may control the power management module to apply a preset first application voltage to a plurality of light emitting elements in the first operation mode. The first voltage may be set to be less than a reference voltage (e.g., a battery application voltage 5V). According to an embodiment, the processor 210 may control at least one optical sensor to switch the first light emitting element outputting the first light (e.g., IR) and the second light emitting element outputting the second light (e.g., green) to which the first application voltage is applied to an activated state (on state). According to an embodiment, the wearing of the electronic device 201 may be detected based on at least a portion (e.g., an IR signal corresponding to a second phase period (phase 2) in FIG. 6A) of the first light reflected from the user's body part during a specified period, and the first biometric information (e.g., the heart rate of the user) may be obtained based on at least a portion (e.g., a green signal corresponding to a fifth phase period (phase 5) in FIG. 6A) of the second light reflected from the user's body part during the specified period. For example, as illustrated in FIG. 6A, the processor 210 may set a period in which no signal is measured, corresponding to a first phase period (phase 1) and a period (AMB) in which no signal is measured between the IR signal and the green signal. According to an embodiment, the processor 210 may repeatedly measure the first biometric information in the first operation mode during the specified period (e.g., a pulse repetition frequency (PRF) cycle of 40 ms), as illustrated in FIG. 6A, and the first light emitting element and the second light emitting element may be activated at different timings to emit light (in the order of IR and green). For example, the processor 210 may perform the first operation mode continuously or during the specified period in normal times, continuously identify a health condition by recording and monitoring heart rate changes in the daily life of the user, and predict a health risk status by identifying a heart rate equal to or greater than a reference heart rate or a heart rate equal to or less than the reference heart rate.

Referring to FIG. 6B, upon occurrence of a specified event, the processor 210 of the electronic device 201 according to an embodiment may identify that a user movement is very low (e.g., sleep), when movement information about the user obtained using the motion sensor is less than the second threshold, and identify the operation mode of the sensor module 230 as the second operation mode (e.g., oxygen saturation measurement mode), further based on environment information (e.g., time) of the user and/or input information. The situation with very low movement (e.g., sleep) may be a situation requiring low accuracy in measuring biometric information. For example, when the current time is night and the user movement is much less than the level of movement for daily life (e.g., the movement information is less than the second threshold), the second operation mode may be identified. According to an embodiment, the processor 210 may control the power management module to apply the first application voltage (e.g., about 3.9V) preset for the second operation mode to at least one optical sensor. According to an embodiment, the at least one processor 210 may control the at least one optical sensor to turn on the first light emitting element outputting the first light (e.g., IR) and the second light emitting element outputting the second light (e.g., green), to which the first application voltage is to be applied. According to an embodiment, the at least one processor 210 may detect the wearing of the electronic device 201 based on at least a portion (e.g., IR signals corresponding to a second phase period (phase 2) and an eighth phase in FIG. 6B) of the first light reflected from the user's body part during a specified period (e.g., a PRF cycle of 10 ms), and obtain first biometric information (e.g., heart rate (HR)) of the user, based on at least a portion (e.g., a green signal corresponding to a fifth phase period (phase 5) in FIG. 6B) of the second light reflected from the user's body part during the specified period. According to an embodiment, the at least one processor 210 may obtain second biometric information (e.g., oxygen saturation (SpO₂)) of the user based on at least a portion (e.g., a red signal corresponding to the fifth phase period (phase 11) in FIG. 6B) of third light reflected from the user's body part during the specified period. For example, as illustrated in FIG. 6B, the processor 210 may set a period in which no signal is measured, corresponding to a first phase period (phase 1) and periods (AMB) in which no signal is measured, among the IR signal, the green signal, and the red signal. According to an embodiment, as illustrated in FIG. 6B, the at least one processor 210 may activate the first light emitting element, the second light emitting element, and the third light emitting element at different timings to output light (e.g., in the order of IR, green, IR, and red) during the specified period (e.g., the PRF cycle of 10 ms). The processor 210 may measure the second biometric information all the time in an on-demand manner or during sleep for measuring sleep apnea and sleep quality, and obtain various indicators (e.g., breathing rate, heart rate, and REM sleep) using the second light (e.g., green). According to an embodiment, the at least one processor 210 may obtain the second biometric information (e.g., oxygen saturation (SpO₂)) with the first light (e.g., IR) and the third light (e.g., red) except the second light (e.g., green).

Referring to FIG. 6C, upon occurrence of a specified event, the processor 210 of the electronic device 201 according to an embodiment may identify that the user has a lot of movement (e.g., exercise) based on movement information obtained using the motion sensor, when the movement information about the user is equal to or greater than the first threshold, which is greater than the second threshold, and identify the operation mode of the sensor module as the third operation mode, further based on environment information (e.g., time) of the user and/or input information in the situation with a lot of movement (e.g., exercise). The situation with a lot of movement (e.g., exercise) may be a situation requiring high accuracy in measuring biometric information. For example, in the case of a time zone in which the user's activity is high and a situation in which the user moves a lot, the third operation mode (e.g., mode for measuring a heart rate using a high current intensity (about 1115 mA)) may be identified. According to an embodiment, the processor 120 may control the power management module to apply a preset second application voltage (e.g., about 4.68V) to the at least one optical sensor in the third operation mode. The second application voltage may be set to a value greater than the reference voltage. According to an embodiment, the processor 210 may control the at least one optical sensor to switch on the first light emitting element outputting the first light (e.g., IR) and the second light emitting element outputting the second light (e.g., green) to which the second application voltage is to be applied. According to an embodiment, the wearing of the electronic device 201 may be detected based on at least a portion (e.g., an IR signal corresponding to a second phase period (phase 2) in FIG. 6B) of the first light reflected from the user's body part during a specified period (e.g., a PRF cycle of 40 ms), and third biometric information (e.g., a heart rate (HR) obtained using a high current intensity (about 115 mA)) of the user may be obtained based on at least a portion (e.g., green signals corresponding to a fifth phase period (phase 5) and an eighth phase period (phase 8) in FIG. 6B) of the second light reflected from the user's body part during the specified period. For example, as illustrated in FIG. 6B, the processor 210 may set a period in which no signal is measured, corresponding to a first phase period (phase 1) and period (AMBs) in which no signal is measured between the IR signal and the green signals. The processor 210 may repeatedly measure the first biometric information in the first operation mode for the specified period (e.g., the PRF cycle of 40 ms), as illustrated in FIG. 6C, and the first light emitting element and the second light emitting element may be activated at different timings to output light (e.g., in the order of IR, green, and green). The processor 210 may calculate, for example, information about a heart rate increase and calorie burning through aerobic exercise by the operation of the third exercise mode, while the user wears the electronic device 201. Since the third operation mode is used when the user exercises, a light emission cycle of the second light may be set longer than those of the other operation modes so that more light reaches the light receiver to prevent the accuracy from decreasing due to shaking, thereby relatively increasing the measurement accuracy through the light received by the light receiver.

According to an embodiment, as illustrated in FIG. 7, the first operation mode and the third operation mode may use, for example, a first current value (e.g. about 60 mA) as the LED current, a forward voltage (Vf) of about 2.6V, a current driver headroom voltage (Vh) of about 0.4V, and a margin voltage (e.g., a voltage added to the voltage Vf+Vh with a margin rate of about 25%) of about 0.9V. According to an embodiment, the at least one processor 210 may set the first application voltage (e.g., about 3.9V) to be applied to at least two light emitting elements to be used based on the LED current and the voltages (Vf, Vh, and margin voltage) set for the first operation mode and the third operation mode. For example, when a voltage value greater than the first application voltage (e.g., about 3.9V) set for the first operation mode and the third operation mode and less than the reference value (e.g., 5V) is applied, there is no difference in performance, but power consumption may increase. Therefore, when the first application voltage (e.g., about 3.9V) set for the first operation mode and the third operation mode is applied to the at least two light emitting elements to be used, power consumption may be reduced.

According to an embodiment, as illustrated in FIG. 7, the second operation mode may use, for example, a second current value (e.g., about 115 mA) as the LED current, a forward voltage (Vf) of about 2.8V, a current driver headroom voltage (Vh) of about 0.8V, and a margin voltage (e.g., a voltage added to the voltage Vf+Vh with a margin rate of about 25%) of about 1.08V. According to an embodiment, the at least one processor 210 may set the second application voltage (e.g., about 4.86V) to be applied to at least two light emitting elements to be used based on the LED current and the voltages (Vf, Vh, and margin voltage) set for the first operation mode and the third operation mode. For example, when a voltage value greater than the second application voltage set for the second operation mode and less than the reference value (e.g., 5V) is applied, there is no difference in performance, but power consumption may increase. Therefore, when the second application voltage set for the second operation mode is applied to the at least two light emitting elements to be used, power consumption may be reduced.

According to an embodiment, the at least one processor 210 may set an application voltage based on an LED current and voltages (Vf, Vh, and margin voltage) for each operation mode, as illustrated in FIG. 7. Current consumed by a light emitting element, such as a light emitting diode (LED), may be identified by considering current flowing through the light emitting element, a pulse width, and a sampling cycle, and the product of the consumed current of the light emitting element and the application voltage of the light emitting element may be identified as power consumption.

In an embodiment, the main components of the electronic device have been described above in the context of the electronic device 201 of FIG. 2. However, in various embodiments, all of the components illustrated in FIG. 2 are not essential components, and the electronic device 201 may be implemented with more or fewer components than the illustrated components. Further, the positions of the main components of the electronic device 201 described above with reference to FIG. 2 may be changed according to various embodiments.

According to an embodiment, an electronic device (e.g., the electronic device of FIG. 1 or the electronic device 201 of FIG. 2, FIG. 3A, and FIG. 3B) may include at least one optical sensor (e.g., the sensor module 176 of FIG. 1 or the sensor module 230 of FIG. 2), memory (e.g., the memory 130 of FIG. 1 or the memory 240 of FIG. 2) storing instructions, and at least one processor (e.g., the processor 120 of FIG. 1 or the processor 210 of FIG. 2).

According to an embodiment, the instructions may be configured to, when executed by the at least one processor, cause the electronic device to set a plurality of operation modes for the sensor module and operation information for each of the plurality of operation modes.

According to an embodiment, the instructions may be configured to, when executed by the at least one processor, cause the electronic device to, based on occurrence of a specified event, identify an operation mode to be used,

According to an embodiment, the instructions may be configured to, when executed by the at least one processor, cause the electronic device to, based on the operation information set for the identified operation mode, identify a voltage to be applied to the plurality of light emitting elements and output the identified voltage. The identified voltage may be less than a reference voltage.

According to an embodiment, the instructions may be configured to, when executed by the at least one processor, cause the electronic device to obtain biometric information based on light irradiated from each of the plurality of light emitting elements to which the identified voltage is applied.

According to an embodiment, the sensor module may include a light receiving element configured to receive at least a portion of light reflected from a body part, a measurement module configured to detect an optical signal by converting the light received by the light receiving element into an electrical signal, and a power management circuit (e.g., the power management module 188 of FIG. 1 or the power management module 220 of FIG. 2) configured to apply the identified voltage to the plurality of light emitting elements.

According to an embodiment, the specified event may include an event based on detection of wearing of the electronic device, an input for requesting measurement of biometric information, a specified time, or execution of a specific application.

According to an embodiment, the instructions may be configured to, when executed by the at least one processor, cause the electronic device to, based on identifying a first operation mode requiring low measurement accuracy of the biometric information, control the sensor module to output a first application voltage set for the first operation mode to the plurality of light emitting elements. The first application voltage may be set to be less than the reference voltage,

According to an embodiment, the sensor module may be controlled to activate the first light emitting element configured to output first light and the second light emitting element configured to output second light (e.g., green), to which the first application voltage is to be applied,

According to an embodiment, the instructions may be configured to, when executed by the at least one processor, cause the electronic device to obtain a first optical signal based on at least a portion of the first light reflected from the body part of a user during a specified period, identify wearing of the electronic device based on the first optical signal, obtain a second optical signal based on at least a portion of the second light reflected from the body part of the user during the specified period, and obtain first biometric information of the user based on the second optical signal.

According to an embodiment, the instructions may be configured to, when executed by the at least one processor, cause the electronic device to, based on identifying a second operation mode requiring low measurement accuracy of the biometric information, control the sensor module to output the first application voltage set for the second operation mode to the plurality of light emitting elements, control the sensor module to activate the first light emitting element configured to output the first light, the second light emitting element configured to output the second light, and the third light emitting element configured to output third light, to which the first application voltage is to be applied, obtain the first optical signal based on at least a portion of the first light reflected from the body part of the user during a specified period, identify wearing of the electronic device based on the first optical signal, obtain the second optical signal based on at least a portion of the second light reflected from the body part of the user during the specified period, obtain the first biometric information of the user based on the second optical signal, and obtain second biometric information of the user based on at least a portion of the third light reflected from the body part of the user during the specified period.

According to an embodiment, the instructions may be configured to, when executed by the at least one processor, cause the electronic device to, based on identifying a third operation mode requiring high measurement accuracy of the biometric information, control the sensor module to output a second application voltage set for the third operation mode to the plurality of light emitting elements. The second application voltage may be set to be greater than the first application voltage set for another mode and less than the reference voltage. According to an embodiment, the instructions may be configured to, when executed by the at least one processor, cause the electronic device to control the at least one sensor to activate the first light emitting element configured to output the first light and the second light emitting element configured to output the second light, to which the second application voltage is to be applied. A current value for the second light emitting element may be set to be greater than a current value set for the first operation mode. According to an embodiment, the instructions may be configured to, when executed by the at least one processor, cause the electronic device to obtain the first optical signal based on at least a portion of the first light reflected from the body part of the user during a specified period, identify wearing of the electronic device based on the first optical signal, obtain a plurality of second optical signals based on at least a portion of the second light reflected from the body part of the user at different timings during the specified period, and obtain third biometric information based on the plurality of second optical signals.

According to an embodiment, the sensor module may further include a motion sensor. According to an embodiment, the instructions may be configured to, when executed by the at least one processor, cause the electronic device to obtain movement information about the user using the motion sensor, and identify the operation mode based on the movement information.

According to an embodiment, the electronic device may further include a display and a communication module. According to an embodiment, the instructions may be configured to, when executed by the at least one processor, cause the electronic device to control the display to display the biometric information and a guidance message generated based on the biometric information, and control the communication module to transmit the biometric information and the guidance message generated based on the biometric information to an external electronic device.

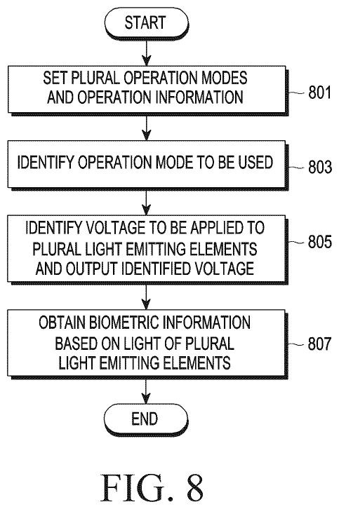

FIG. 8 is a diagram illustrating an exemplary method of operating an electronic device according to an embodiment.

In the embodiment, each operation may be performed sequentially, but not necessarily. For example, the order of each operation may be changed, and at least two operations may be performed in parallel.

According to an embodiment, it may be understood that operations 801 to 807 are performed in a processor (e.g., the processor 120 of FIG. 1 or the processor 210 of FIG. 2) of an electronic device (e.g., the electronic device 101 of FIG. 1 or the electronic device 201 of FIG. 2, FIG. 3A, and FIG. 3B).

Referring to FIG. 8, in operation 801, the electronic device (e.g., the electronic device 101 of FIG. 1 or the electronic device 201 of FIG. 2, FIG. 3A, and FIG. 3B) according to an embodiment may set a plurality of operation modes for a sensor module (e.g., the sensor module 230 of FIGS. 3B and 4) and operation information for each of the plurality of operation modes. The electronic device may preset the plurality of operation modes and configure the operation information before performing an operation of measuring biometric information or upon request of a user.

In operation 803, the electronic device according to an embodiment may identify an operation mode to be used, based on occurrence of a specified event. According to an embodiment, the electronic device may identify an operation mode to be used for operating the sensor module, based on movement information about the user, environment information, and/or input information. The specified event may be an event based on detection of wearing of the electronic device, an input for requesting measurement of biometric information, a specified time, or execution of a specific application.

In operation 805, the electronic device according to an embodiment may identify an application voltage output from a power management module to a plurality of light emitting elements based on the operation information configured for the identified operation mode, and output the identified application voltage to the plurality of light emitting elements.

In operation 807, the electronic device according to an embodiment may activate (turn on) at least two light emitting elements included in a light emitter (e.g., the light emitter 411 of FIG. 4) of a sensor module to which a changed application voltage is applied during a specified period. The electronic device may obtain biometric information based on light emitted from the activated light emitting elements.

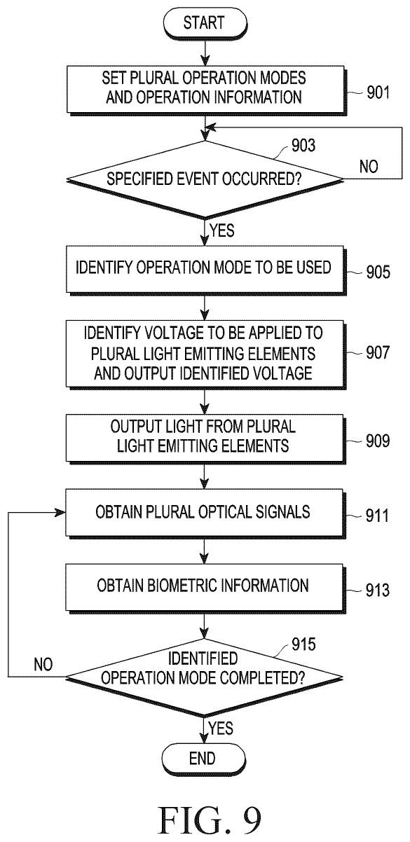

FIG. 9 is a diagram illustrating an exemplary method of operating an electronic device according to an embodiment.

Referring to FIG. 9, in operation 901, the electronic device (e.g., the electronic device 101 of FIG. 1 or the electronic device 201 of FIG. 2, FIG. 3A, and FIG. 3B) according to an embodiment may set a plurality of operation modes for a sensor module (e.g., the sensor module 230 of FIG. 2, FIG. 3B, and FIG. 4) and operation information for each of the plurality of operation modes. The electronic device may preset the plurality of operation modes and configure the operation information before performing an operation of measuring biometric information or upon request of a user. The operation information may include information about a voltage to be applied to the plurality of light emitting elements, information (e.g., the types of light emitting elements (e.g., LEDs) and/or an operating current (LED current or drive current) of each light emitting element and operating voltages (Vf, Vh, and margin voltage)) about the light emitting elements to be used among light emitting elements of different wavelengths included in a sensor module (e.g., the sensor module 176 of FIG. 1 or the sensor module 230 of FIG. 2 and FIG. 3). According to an embodiment, the electronic device may set operation modes based on the types of biometric information and the user's situations (e.g., movement, time, and environment). For example, the electronic device may set a first operation mode for measuring first biometric information (e.g., heart rate (HR)) in a situation (e.g., normal times) where the user's movement is less than a first threshold and equal to or greater than a second threshold, a second operation mode for measuring second biometric information (e.g., heart rate (HR) with a large current intensity) in a situation (e.g., exercise) where the user's movement is equal to or greater than the first threshold, and a third operation mode for measuring third biometric information (e.g., blood oxygen saturation) in a situation (e.g., sleep) where the user's movement is so small as to be less than the second threshold. In addition, the electronic device may further set an operation mode for measuring biometric information such as stress and blood pressure.

In operation 903, the electronic device according to an embodiment may identify whether a specified event has occurred. The specified event may include, but is not limited to, an event based on detection of wearing of the electronic device, an input for requesting measurement of biometric information, a specified time, or execution of a specific application. When the specified event occurs as a result of the identification, the electronic device may perform operation 905, and when the specified event does not occur, the electronic device may repeatedly perform operation 903.

In operation 905, the electronic device according to an embodiment may identify an operation mode to be used based on the occurrence of the specified event. According to an embodiment, when the specified event occurs, the electronic device may identify an operation mode to be used to operate the sensor module based on movement information about the user, environment information, and/or input information. As described herein, the specified event may be an event based on detection of wearing of the electronic device, an input for requesting measurement of biometric information, a specified time, or execution of a specific application. According to an embodiment, when the electronic device identifies a situation in which the user's movement is low to the level of movement for daily life based on movement information obtained using a motion sensor, the electronic device may identify the operation mode of the sensor module as the first operation mode. According to an embodiment, when the electronic device identifies a situation in which the user's movement is very low, such as a sleeping state, the electronic device may identify the operation mode of the sensor module as the second operation mode. According to an embodiment, when the electronic device identifies a situation in which the user moves a lot, such as an exercise state, the electronic device may identify the operation mode of the sensor module as the third operation mode.

In operation 907, the electronic device according to an embodiment may identify an application voltage output from the power management module to a plurality of light emitting elements as an application voltage set for the identified operation mode, based on the operation information configured for the identified operation mode, and output the identified application voltage to the plurality of light emitting elements. According to an embodiment, when the electronic device identifies the operation mode of the sensor module as the first operation mode or the third operation mode, the electronic device may apply a first application voltage (e.g., about 3.9V) set for the first operation mode or the third operation mode to the plurality of light emitting elements. According to an embodiment, when the electronic device identifies the operation mode of the sensor module as the third operation mode, the electronic device may apply a second application voltage (e.g., about 4.68V) set for the third operation mode to the plurality of light emitting elements.

In operation 909, the electronic device according to an embodiment may activate (turn on) at least two light emitting elements included in a light emitter (e.g., the light emitter 411 of FIG. 4) of the sensor module to which the changed application voltage is applied during a specified period. The electronic device may start the operation of the sensor module to measure biometric information using the activated light emitting elements, irradiate light from the at least two light emitting elements activated during the specified period to the user's body part, and receive at least a portion of light reflected from the body part through a light receiver (e.g., the light receiver 413 of FIG. 4) included in the sensor module. According to an embodiment, the electronic device may activate a first light emitting element and a second light emitting element in the first operation mode. The first light emitting element may output first light (e.g., IR) for detecting the user's wearing of the electronic device as reference light, and the second light emitting element may output second light (e.g., green) for measuring first biometric information (e.g., heart rate). In the first operation mode, the first light and the second light may be output (e.g., in the order of IR and green) at different timings during a specified period (e.g., a PRF cycle of 40 ms). According to an embodiment, the electronic device may activate the first light emitting element, the second light emitting element, and a third light emitting element in the second operation mode. The first light emitting element may output the first light (e.g., IR) for detecting the user's wearing of the electronic device as the reference light, and the second light emitting element may output the second light (e.g., green) for measuring the first biometric information (e.g., heart rate). The third light emitting element may output third light (e.g., red) for measuring second biometric information (e.g., oxygen saturation). In the second operation mode, the first light, the second light, and the third light may be output (e.g., in the order of IR, green, IR, and red) at different timings during a specified period (e.g., a PRF cycle of 10 ms). According to an embodiment, the electronic device may activate the first light emitting element and the second light emitting element in the third operation mode. The first light emitting element may output the first light (e.g., IR) for detecting the user's wearing of the electronic device as the reference light, and the second light emitting element may output the second light (e.g., green) for measuring the third biometric information (e.g., heart rate) at a current intensity (e.g., about 1115 mA or about 230 mA) that is about twice as high as in the first operation mode. For example, the second light may be output at a cycle that is twice as long as in the first operation mode. In the third operation mode, the first light and the second light may be output at different timings (e.g., in the order of IR, green, and green) during a specified period (e.g., a PRF cycle of 40 ms).

In operation 911, the electronic device according to an embodiment may obtain a plurality of optical signals of different wavelengths from light received through the light receiver of the sensor module. According to an embodiment, in the first operation mode, the electronic device may receive at least portions of the first light and the second light output from the first light emitting element and the second light emitting element, respectively, obtain a first optical signal by electrically converting at least a portion of the received first light, and obtain a second optical signal by electrically converting at least a portion of the second light. According to an embodiment, in the second operation mode, the electronic device may receive at least portions of the first light, the second light, and the third light output from the first light emitting element, the second light, and the third light, respectively, obtain a first optical signal by electrically converting at least a portion of the received first light, obtain a second optical signal electrically converted at least a portion of the second light, and obtain a third optical signal by electrically converting at least a portion of the third light. According to an embodiment, in the third operation mode, the electronic device may receive the first light output from each of the first light emitting element and the second light emitting element, repeatedly receive at least portions of second light on channels of different timings, obtain a first optical signal by electrically converting at least a portion of the received first light, and obtain second optical signals by electrically converting at least portions of the second light received on different channels, during a specified period.

In operation 913, the electronic device according to an embodiment may obtain biometric information based on a plurality of optical signals obtained during the specified period in the identified operation mode.

When performing operation 913, according to an embodiment, the electronic device may detect the wearing of the electronic device based on the first optical signal obtained during the specified period, and obtain the first biometric information (e.g., heart rate (HR)) of the user based on the second optical signal obtained during the specified period, according to the execution of the first operation mode. The first operation mode for measuring the first biometric information may be executed continuously or during the specified period, for example. The electronic device may continuously identify the user's health condition by executing the first operation mode and recording and monitoring a change in the user's heart rate in the user's daily life, and may identify a heart rate equal to or less than a reference heart rate or a heart rate or equal to or greater than to the reference heart rate to predict the health risk state.

When performing operation 913, according to an embodiment, the electronic device may detect the wearing of the electronic device based on the first optical signal obtained during the specified period, obtain the first biometric information (e.g., heart rate (HR)) of the user based on the second optical signal obtained during the specified period, and obtain the second biometric information (e.g., oxygen saturation (SpO₂)) of the user based on the third optical signal obtained during the specified period, according to the execution of the second operation mode. In the second operation mode, the electronic device may measure the second biometric information continuously in an on-demand manner, for example, or measure the second biometric information during sleep to measure sleep apnea and sleep quality, and may obtain various indicators (e.g., breathing rate, heart rate, rapid eye movement (REM) sleep) using the second light (e.g., green).

When performing operation 913, according to an embodiment, the electronic device may detect the wearing of the electronic device based on the first optical signal obtained during the specified period, and obtain highly accurate third biometric information (e.g., heart rate (HR)) based on the second optical signal obtained during the specified period, according to the execution of the third operation mode. In the third operation mode, the electronic device may calculate, for example, information about a heart rate increase and calorie burning through aerobic exercise, while the user is wearing the electronic device 201. Since the third operation mode is used when the user exercises with a lot of movement, the light emission cycle of the second light may be set longer than those of other operation modes so that a lot of light reaches the light receiver to prevent the accuracy from decreasing due to shaking, thereby relatively increasing the measurement accuracy through the light received from the light receiver through a channel.

In operation 915, the electronic device may identify whether the identified operation mode has been completely performed. As a result of the identification, when the identified operation mode is completed, the operation is terminated, and when the identified operation mode is not completed, operation 911 may be performed again.

After performing the operation method of FIG. 9 described above, when the operation of measuring biometric information is terminated or the operation mode is changed, the activated light emitting elements of the at least one optical sensor may be switched to a deactivated (off) state.

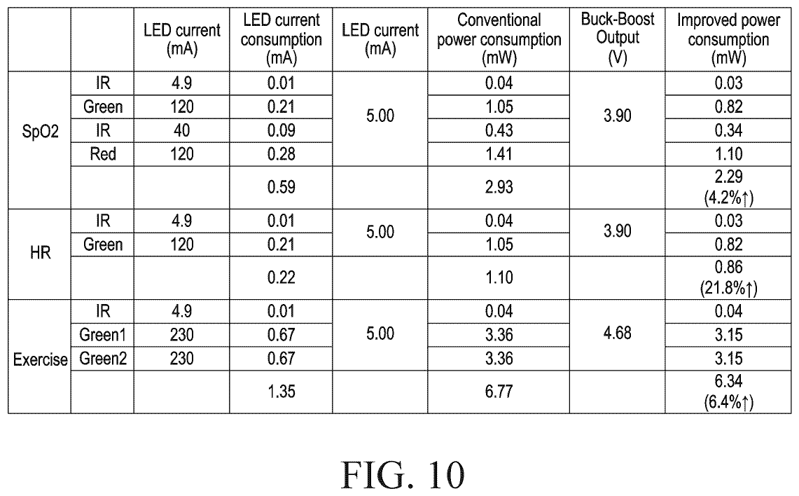

FIG. 10 is a diagram illustrating an example of improved power consumption of an electronic device according to an embodiment.

As illustrated in FIG. 10, power consumption may be reduced compared to conventional power consumption by applying an application voltage set for each operation mode to a plurality of light emitting elements included in the sensor module according to the operation method of FIGS. 8 and 9 described above. For example, as illustrated in FIG. 10, when the current and current consumption of the second light emitting element (e.g., a LED) that outputs the second light (e.g., green) is about 120 mA and about 0.21, respectively, and the reference voltage is 5V in the first operation mode (HR) and the third operation mode (SpO₂), the conventional power consumption may be about 1.05 mW, and when a changed application voltage is about 3.9V in the first operation mode and the third operation mode, the power consumption may be improved to about 0.82 mW. In the first operation mode, total power consumption according to a light output operation (e.g., IR and green) of all light emitting elements during a specified period may be improved to about 0.86 mW from conventional power consumption of about 1.10 mW, thus by approximately 21.8%. As illustrated in FIG. 9, in the second operation mode (SpO₂), total power consumption according to a light output operations (e.g., IR, green, IR, and red) of all light emitting elements during a specified period may be improved to approximately 2.29 mW from conventional power consumption of approximately 2.93 mW, thus by approximately 4.2%. As illustrated in FIG. 9, in the third operation mode (exercise), total power consumption according to a light output operations (e.g., IR, green 1, and green 2) of all light emitting elements to which an application voltage of approximately 4.68V is applied during a specified period is about 6.34 mW, which is improved from conventional power consumption of approximately 6.77 mW by approximately 6.4%.

According to an embodiment, a method of operating an electronic device (e.g., the electronic device 101 of FIG. 1 or the electronic device 201 of FIG. 2, FIG. 3A, and FIG. 3B) may include setting a plurality of operation modes for a sensor module (e.g., the sensor module 230 of FIGS. 2, 3B, and 4) of the electronic device and operation information for each of the plurality of operation modes.

According to an embodiment, the method may include, based on occurrence of a specified event, identifying an operation mode to be used.

According to an embodiment, the method may include, based on the operation information set for the identified operation mode, identifying a voltage to be applied to the plurality of light emitting elements and outputting the identified voltage. According to an embodiment, the identified voltage may be less than a reference voltage.

According to an embodiment, the method may include obtaining biometric information based on light irradiated from each of the plurality of light emitting elements to which the identified voltage is applied. According to an embodiment, the plurality of light emitting elements may include a first light emitting element and at least one of a second light emitting element or a third light emitting element. According to an embodiment, the specified event may include an event based on detection of wearing of the electronic device, an input for requesting measurement of biometric information, a specified time, or execution of a specific application.

According to an embodiment, outputting the identified voltage may include, based on identifying a first operation mode requiring low measurement accuracy of the biometric information, outputting a first application voltage set for the first operation mode to the plurality of light emitting elements, and activating the first light emitting element outputting first light and the second light emitting element outputting second light (Green), to which the first application voltage is to be applied. According to an embodiment, the first application voltage may be set to be less than the reference voltage,

According to an embodiment, obtaining the biometric information may include obtaining a first optical signal based on at least a portion of the first light reflected from the body part of a user during a specified period, identifying wearing of the electronic device based on the first optical signal, obtaining a second optical signal based on at least a portion of the second light reflected from the body part of the user during the specified period, and obtaining first biometric information of the user based on the second optical signal.

According to an embodiment, outputting the identified voltage may include, based on identifying a second operation mode requiring low measurement accuracy of the biometric information, outputting the first application voltage set for the second operation mode to the plurality of light emitting elements, and activating the first light emitting element configured to output the first light, the second light emitting element configured to output the second light, and the third light emitting element configured to output third light, to which the first application voltage is to be applied.

According to an embodiment, obtaining the biometric information may include obtaining the first optical signal based on at least a portion of the first light reflected from the body part of the user during a specified period, identifying wearing of the electronic device based on the first optical signal, obtaining the second optical signal based on at least a portion of the second light reflected from the body part of the user during the specified period, obtaining the first biometric information of the user based on the second optical signal, and obtaining second biometric information of the user based on at least a portion of the third light reflected from the body part of the user during the specified period.

According to an embodiment, outputting the identified voltage may include, based on identifying a third operation mode requiring high measurement accuracy of the biometric information, outputting a second application voltage set for the third operation mode to the plurality of light emitting elements, and activating the first light emitting element configured to output the first light and the second light emitting element configured to output the second light, to which the second application voltage is to be applied.

According to an embodiment, the second application voltage may be set to be greater than the first application voltage set for another mode and less than the reference voltage.

According to an embodiment, a current value for the second light emitting element may be set to be greater than a current value set for the first operation mode.

According to an embodiment, obtaining the biometric information may include obtaining the first optical signal based on at least a portion of the first light reflected from the body part of the user during a specified period, identifying wearing of the electronic device based on the first optical signal, obtaining a plurality of second optical signals based on at least a portion of the second light reflected from the body part of the user at different timings during the specified period, and obtaining third biometric information based on the plurality of second optical signals.

According to an embodiment, identifying the operation mode to be used may include obtaining movement information about the user using a motion sensor, and identifying the operation mode based on the movement information.

According to an embodiment, the method may further include displaying the biometric information and a guidance message generated based on the biometric information on the display 250 of the electronic device, and transmitting the biometric information and the guidance message generated based on the biometric information to an external electronic device through the communication module 260 of the electronic device.

According to an embodiment, in a non-transitory storage medium storing a program, the program may include executable instructions that, when executed by at least one processor (e.g., the processor 120 of FIG. 1 or the processor 210 of FIG. 2) of an electronic device (e.g., the electronic device 101 of FIG. 1 or the electronic device 201 of FIG. 2, FIG. 3A, and FIG. 3B), cause the electronic device to set a plurality of operation modes for a sensor module (e.g., the sensor module 176 of FIG. 1 or the sensor module 230 of FIG. 2, FIG. 3B, and FIG. 4) of the electronic device and operation information for each of the plurality of operation modes, based on occurrence of a specified event, identify an operation mode to be used, based on the operation information set for the identified operation mode, identify a voltage to be applied to the plurality of light emitting elements, output the identified voltage, wherein the identified voltage is less than a reference voltage, and obtain biometric information based on light irradiated from each of the plurality of light emitting elements to which the identified voltage is applied.

According to an embodiment, in the non-transitory storage medium, identifying the operation mode to be used may include obtaining movement information about a user using a motion sensor, identifying a first operation mode or a second operation mode requiring low measurement accuracy of the biometric information based on the movement information, and identifying a third operation mode requiring high measurement accuracy of the biometric information based on the movement information. According to an embodiment, an application voltage of at least one optical sensor may be set to a first application voltage in the first operation mode and the second operation mode, and the voltage to be applied to the plurality of light emitting elements may be set to a second application voltage in the third operation mode. According to one embodiment, the second application voltage may be set to be less than the reference voltage and greater than the first application voltage.

According to an embodiment of the disclosure, as the electronic device measures biometric information by applying an application voltage set for each operation mode and operating the sensor module, the electronic device may reduce power consumption while maintaining the performance of the sensor module. In addition, various effects that are directly or indirectly achieved from the disclosure may be provided.

The embodiments of the disclosure are provided for the purpose of describing and understanding the disclosed technical contents, and do not limit the technical scope of the disclosure. Therefore, the scope of the disclosure should be interpreted to include all modifications or various other embodiments based on the technical idea of the disclosure.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B”, “at least one of A and B”, “at least one of A or B”, “A, B, or C,” at least one of A, B, and C, and “at least one of A, B, or C”, may include any one of, or all possible combinations of the items enumerated together in a corresponding one of the phrases. As used herein, such terms as “1^{st}” and “2^{nd}”, or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, logic, logic block, part, or circuitry. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

1. An electronic device comprising:
a sensor module including a plurality of light emitting elements and at least one optical sensor, wherein the plurality of light emitting elements include a first light emitting element and at least one of a second light emitting element or a third light emitting element;
memory storing instructions; and
at least one processor,
wherein the instructions are configured to, when executed by the at least one processor, cause the electronic device to:
set a plurality of operation modes for the sensor module and set operation information for each of the plurality of operation modes,
based on occurrence of a specified event, identify an operation mode to be used,
based on the operation information set for the identified operation mode, identify a voltage to be applied to the plurality of light emitting elements and output the identified voltage, wherein the identified voltage is less than a reference voltage, and
obtain biometric information based on light irradiated from each of the plurality of light emitting elements to which the identified voltage is applied.

2. The electronic device of claim 1, wherein the sensor module includes:
a light receiving element configured to receive at least a portion of light reflected from a body part;
a measurement module configured to detect an optical signal by converting the light received by the light receiving element into an electrical signal; and
a power management circuit configured to apply the identified voltage to the plurality of light emitting elements, and
wherein the specified event includes an event based on detection of wearing of the electronic device, an input for requesting measurement of biometric information, a specified time, or execution of a specific application.

3. The electronic device of claim 1, wherein the instructions are configured to, when executed by the at least one processor, cause the electronic device to:
based on identifying a first operation mode requiring low measurement accuracy of the biometric information, control the sensor module to output a first application voltage set for the first operation mode to the plurality of light emitting elements, wherein the first application voltage is set to be less than the reference voltage,
control the sensor module to activate the first light emitting element configured to output first light and the second light emitting element configured to output second light, to which the first application voltage is to be applied,
obtain a first optical signal based on at least a portion of the first light reflected from the body part of a user during a specified period, and identify wearing of the electronic device based on the first optical signal, and
obtain a second optical signal based on at least a portion of the second light reflected from the body part of the user during the specified period, and obtain first biometric information of the user based on the second optical signal.

4. The electronic device of claim 1, wherein the instructions are configured to, when executed by the at least one processor, cause the electronic device to:
based on identifying a second operation mode requiring low measurement accuracy of the biometric information, control the sensor module to output the first application voltage set for the second operation mode to the plurality of light emitting elements,
control the sensor module to activate the first light emitting element configured to output the first light, the second light emitting element configured to output the second light, and the third light emitting element configured to output third light, to which the first application voltage is to be applied,
obtain a first optical signal based on at least a portion of the first light reflected from the body part of the user during a specified period, and identify wearing of the electronic device based on the first optical signal,
obtain a second optical signal based on at least a portion of the second light reflected from the body part of the user during the specified period, and obtain the first biometric information of the user based on the second optical signal, and
obtain second biometric information of the user based on at least a portion of the third light reflected from the body part of the user during the specified period.

5. The electronic device of claim 1, wherein the instructions are configured to, when executed by the at least one processor, cause the electronic device to:
based on identifying a third operation mode requiring high measurement accuracy of the biometric information, control the sensor module to output a second application voltage set for the third operation mode to the plurality of light emitting elements, wherein the second application voltage is set to be greater than the first application voltage set for another mode and less than the reference voltage,
control the at least one sensor to activate the first light emitting element configured to output the first light and the second light emitting element configured to output the second light, to which the second application voltage is to be applied, wherein a current value for the second light emitting element is set to be greater than a current value set for the first operation mode,
obtain a first optical signal based on at least a portion of the first light reflected from the body part of the user during a specified period, and identify wearing of the electronic device based on the first optical signal, and
obtain a plurality of second optical signals based on at least a portion of the second light reflected from the body part of the user at different timings during the specified period, and obtain third biometric information based on the plurality of second optical signals.

6. The electronic device of claim 1, wherein the sensor module further includes a motion sensor, and
wherein the instructions are configured to, when executed by the at least one processor, cause the electronic device to:
obtain movement information about the user using the motion sensor, and
identify the operation mode based on the movement information.

7. The electronic device of claim 1, further including:
a display; and
a communication module,
wherein the instructions are configured to, when executed by the at least one processor, cause the electronic device to:
control the display to display the biometric information and a guidance message generated based on the biometric information, and
control the communication module to transmit the biometric information and the guidance message generated based on the biometric information to an external electronic device.

8. A method of operating an electronic device, comprising:
setting a plurality of operation modes for a sensor module of the electronic device and setting operation information for each of the plurality of operation modes;
based on occurrence of a specified event, identifying an operation mode to be used;
based on the operation information set for the identified operation mode, identifying a voltage to be applied to the plurality of light emitting elements and outputting the identified voltage, wherein the identified voltage is less than a reference voltage; and
obtaining biometric information based on light irradiated from each of the plurality of light emitting elements to which the identified voltage is applied,
wherein the plurality of light emitting elements include a first light emitting element and at least one of a second light emitting element or a third light emitting element.

9. The method of claim 8, wherein the specified event includes an event based on detection of wearing of the electronic device, an input for requesting measurement of biometric information, a specified time, or execution of a specific application.

10. The method of claim 8, wherein outputting the identified voltage includes:
based on identifying a first operation mode requiring low measurement accuracy of the biometric information, outputting a first application voltage set for the first operation mode to the plurality of light emitting elements, wherein the first application voltage is set to be less than the reference voltage; and
activating the first light emitting element configured to output first light and the second light emitting element configured to output second light, to which the first application voltage is to be applied, and
wherein obtaining the biometric information includes:
obtaining a first optical signal based on at least a portion of the first light reflected from the body part of a user during a specified period, and identifying wearing of the electronic device based on the first optical signal; and
obtaining a second optical signal based on at least a portion of the second light reflected from the body part of the user during the specified period, and obtaining first biometric information of the user based on the second optical signal.

11. The method of claim 8, wherein outputting the identified voltage includes:
based on identifying a second operation mode requiring low measurement accuracy of the biometric information, outputting the first application voltage set for the second operation mode to the plurality of light emitting elements; and
activating the first light emitting element configured to output the first light, the second light emitting element configured to output the second light, and the third light emitting element configured to output third light, to which the first application voltage is to be applied, and
wherein obtaining the biometric information includes:
obtaining a first optical signal based on at least a portion of the first light reflected from the body part of the user during a specified period, and identifying wearing of the electronic device based on the first optical signal;
obtaining a second optical signal based on at least a portion of the second light reflected from the body part of the user during the specified period, and obtaining the first biometric information of the user based on the second optical signal; and
obtaining second biometric information of the user based on at least a portion of the third light reflected from the body part of the user during the specified period.

12. The method of claim 8, wherein outputting the identified voltage includes:
based on identifying a third operation mode requiring high measurement accuracy of the biometric information, outputting a second application voltage set for the third operation mode to the plurality of light emitting elements; and
activating the first light emitting element configured to output the first light and the second light emitting element configured to output the second light, to which the second application voltage is to be applied,
wherein the second application voltage is set to be greater than the first application voltage set for another mode and less than the reference voltage,
wherein a current value for the second light emitting element is set to be greater than a current value set for the first operation mode, and
wherein obtaining the biometric information includes:
obtaining a first optical signal based on at least a portion of the first light reflected from the body part of the user during a specified period, and identifying wearing of the electronic device based on the first optical signal; and
obtaining a plurality of second optical signals based on at least a portion of the second light reflected from the body part of the user at different timings during the specified period, and obtaining third biometric information based on the plurality of second optical signals.

13. The method of claim 8, wherein identifying the operation mode to be used includes:
obtaining movement information about the user using a motion sensor; and
identifying the operation mode based on the movement information.

14. The method of claim 8, further including:
displaying the biometric information and a guidance message generated based on the biometric information on a display of the electronic device; and
transmitting the biometric information and the guidance message generated based on the biometric information to an external electronic device through a communication module of the electronic device.

15. A non-transitory storage medium storing a program including executable instructions that, when executed by at least one processor of an electronic device, cause the electronic device to:
set a plurality of operation modes for a sensor module of the electronic device and set operation information for each of the plurality of operation modes;
based on occurrence of a specified event, identify an operation mode to be used;
based on the operation information set for the identified operation mode, identify a voltage to be applied to the plurality of light emitting elements and output the identified voltage, wherein the identified voltage is less than a reference voltage; and
obtain biometric information based on light irradiated from each of the plurality of light emitting elements to which the identified voltage is applied,
wherein the plurality of light emitting elements include a first light emitting element and at least one of a second light emitting element or a third light emitting element.

16. An electronic device comprising:
a sensor module including a plurality of light emitting elements and at least one optical sensor, wherein the plurality of light emitting elements include a first light emitting element configured to emit a first wavelength of light and at least one of a second light emitting element configured to emit a second wavelength of light or a third light emitting element configured to emit a third wavelength of light;
memory storing instructions; and
at least one processor configured to execute the instructions to:
set a plurality of operation modes for the sensor module and operation information for each operation mode;
identify an operation mode to be used based on occurrence of a specified event;
based on the operation information for the identified operation mode, select a subset of the plurality of light emitting elements to be activated;
identify a current to be applied to each selected light emitting element based on the identified operation mode;
apply the identified current to the selected light emitting elements; and
obtain biometric information based on light emitted from the selected light emitting elements and received by the at least one optical sensor.

17. The electronic device of claim 16, wherein the plurality of operation modes include a first operation mode associated with a sedentary state, a second operation mode associated with a sleep state, and a third operation mode associated with an exercise state.

18. The electronic device of claim 17, wherein each mode is associated with a corresponding current level and a corresponding subset of the plurality of light emitting elements.

19. The electronic device of claim 16, wherein:
in the first operation mode, the selected light emitting element comprises a green light emitting diode (LED) and the applied current is a first current level;
in the second operation mode, the selected light emitting elements comprise a red LED and an infrared LED, and the applied current is a second current level lower than the first current level; and
in the third operation mode, the selected light emitting element comprises a green LED and the applied current is a third current level higher than the first and second current levels, the third current applied to the green LED during a light emission cycle that is longer than a light emission cycle of the first operation mode and the second operation mode.

20. The electronic device of claim 16, wherein the current applied to the selected light emitting elements is determined based on a forward voltage, a headroom voltage, and a margin voltage configured for the identified operation mode.
